(19) United States
(12) Patent Application Publication
(10) Pub. No.: US 2025/0161538 A1
(43) Pub. Date: May 22, 2025

(54) POLYMER-BASED MATERIAL WITH ANTI-BACTERIAL PROPERTIES WITH IMPROVED MECHANICAL STRENGTH FOR DENTAL APPLICATIONS

(71) Applicants: Esra TUNALI, Canakkale (TR); Onder ALBAYRAK, Mersin (TR); Seniz KARACAY, Istanbul (TR); Mustafa TUNALI, Canakkale (TR); Ersin YILDIRIM, Istanbul (TR)

(72) Inventors: Esra TUNALI, Canakkale (TR); Onder ALBAYRAK, Mersin (TR); Seniz KARACAY, Istanbul (TR); Mustafa TUNALI, Canakkale (TR); Ersin YILDIRIM, Istanbul (TR)

(21) Appl. No.: 18/839,033

(22) PCT Filed: Sep. 26, 2022
(86) PCT No.: PCT/TR2022/051040

§ 371 Date: Aug. 16, 2024

(30) Foreign Application Priority Data
Feb. 24, 2022 (TR) 2022/002642

(51) Int. Cl. A61L 27/54; A61L 27/02; A61L 27/08; A61L 27/12

(52) U.S. Cl. CPC A61L 27/54; A61L 27/025; A61L 27/08; A61L 27/12; A61L 2300/404; A61L 2430/12

(57) ABSTRACT

An agent is provided, where the agent gives antibacterial effect to all kinds of polymer-based appliances, plaques and retainers (retainer; transparent or colored) used in all dentistry and improve their mechanical properties; and the agent relates to materials in the form of powders, suspensions or solutions used in their ingredients, manufacture, coating or surface treatment. In this context, Ca—P-based ceramics, B, graphene, Mg, Se, Sr or their derivatives in powder form, suspensions or solutions containing these powders are used individually or as a mixture.

### CROSS REFERENCE TO THE RELATED APPLICATIONS

This application is the national phase entry of International Application No. PCT/TR2022/051040, filed on Sep. 26, 2022, which is based upon and claims priority to Turkish Patent Application No. 2022/002642, filed on Feb. 24, 2022, the entire contents of which are incorporated herein by reference.

### TECHNICAL FIELD

The invention relates to a material that will provide all kinds of polymer-based appliances, plaques and retainers (clear or colored) used in dental applications with improved mechanical properties and antibacterial effect. The material subject to our patent application will be used directly as an additive material in the production, coating, surface treatment and/or content of all kinds of polymer-based appliances, plaques and retainers used in dentistry.

### BACKGROUND

Tooth deformities occur due to genetic or environmental reasons, and if they are not treated, they cause problems in terms of oral hygiene, oral and jaw health, as well as negatively affect patients psychologically and socially. Retainer treatment is applied after braces are removed in order to protect the corrected teeth after orthodontic treatment for the elimination of tooth deformities and to keep them intact. The treatment, which is generally applied for aesthetic purposes, is now applied due to the necessity of oral and jaw health with the awareness of the patients. In orthodontic treatment, the tendency of the teeth to return to their pre-treatment position is an important problem. Retention is an important stage of orthodontic treatment in which tooth movements are stabilized after active treatment. After the completion of orthodontic treatment, the teeth tend to return to their initial positions due to the tension in the periodontal fibers. Therefore, retention, which is considered the last stage of treatment, is necessary to maintain the desired position of the teeth. Retainers are used to prevent teeth from returning to their former positions until skeletal growth, gingival and periodontal realignment is complete. Retainers are divided into 3 groups as fixed, movable “Hawley” and transparent (essix) depending on both the needs and preferences of the patient and the orthodontist, and they are made of polymer-based material. Factors such as temperature, humidity and elastic deformation have a significant effect on the mechanical properties of the grippers. Therefore, the mechanical properties of these materials may differ between the intraoral environment and room temperature. However, exposure of the polymers to water can cause the material to swell due to water absorption and affect the performance of the retainer. In addition, depending on the glass transition temperature of the polymer used, the material shows brittle or ductile properties at ambient temperature and this affects the mechanical properties.

Studies are carried out in order to obtain improved strength and high wear resistance in Retainers; Gardner et al. The abrasion resistance of three different thermoplastic retainers, namely “Essix C+ (PPC), Invisacryl C (PP) and TR (PETG)”, was investigated. It was determined that polyethylene copolymer (PETG) exhibited higher abrasion resistance than polypropylene (PP) and polypropylene copolymer (PPC). Regarding wear resistance, Raja et al. In the study conducted by “Essix C+ (PPC), Essix ACE (PET), Duran (PETG) and Tru-Train (PETG)” were used. Obtained results, Gardner et al. It supports the work done by; It was concluded that polyethylene copolymer (Essix ACE, Duran and Tru-Train) showed higher abrasion resistance than polypropylene copolymer (Essix C+). It was stated that Essix ACE (PET) had the least wear, and there was no statistically significant difference between Duran (PETG) and Tru-Train (PETG).

Ryokawa et al. “EVA (Bioplast), PE (Copyplast), PETG (Duran), PP (Hardcast), PC (Imprelon‘S’), PET (Essix A+), PPC (Essix C+) and PUR to evaluate the mechanical properties of polymers used in dental applications. They worked on eight different dental thermoplastic products, including “Invisalign”. Depending on the free volume of the material, the water absorption was investigated; PUR (Invisalign) had the highest water absorption capacity, followed by PETG (Duran) and PET (Essix A+), but no significant difference was observed between the two, while PE (Copyplast) had the lowest water absorption rate. stated to have. In the same study, tensile test was performed to examine the mechanical properties before and after thermoforming, and elastic modulus and yield stress were calculated from the obtained stress-strain curves. As a result of the tensile test, it was stated that the highest yield stress was 61.3 MPa, 48.9 MPa and 48.5 MPa for PUR, PETG and PET, respectively, and EVA had the lowest yield strength.

Due to the pressure exerted by the teeth during biting in polymer-based plaques and appliances used in dental applications (bite force can reach up to 70 kgf, especially in the posterior regions), the formation of deformation, cracks and fractures also negatively affects the materials used; The high mechanical strength of such materials provides an advantage in terms of use. In the literature search conducted by us, no studies were found on the use of additives to increase the strength of orthodontic retainers. The negative effects of environmental conditions on mechanical properties are not only encountered in retainers, but also in all kinds of polymer-based appliances, plates and retainers used in dental applications. Patients with bruxism suffer from temporamandibular joint complaints and pain in the jaw and facial muscles; To prevent these complaints and to protect the jaw joint, night plates and splints are made. In cases of partial edentulism and/or complete edentulism, mouth, tooth, jaw and facial prostheses are made to restore the function of the tissue loss. In such cases, it is desired that the polymer-based materials used have high mechanical strength and that they are not affected or less affected by ambient conditions.

In the literature, it is stated that there is an increase in the number of caries-causing bacteria such as S. Mutans and Lactobacillus, and bacteria that can cause gingival disorders such as Porphyromonas Gingivalis, depending on the use of materials used in the correction and function of the mouth, teeth and jaw structure. For this reason, deterioration of the oral microbial flora, gingivitis, white spot lesions and a predisposition to dental caries occur. In addition, halitosis (bad breath) caused by increased bacterial colonization in the mouth is among the main complaints of patients.

There are studies in the literature on additives in order to give antibacterial effects to biomaterials; For this purpose, mostly silver (Ag) is used as an additive material. In addition to Ag, materials such as zinc (Zn), titanium dioxide (TiO₂) are also used to gain antibacterial activity or to increase the antibacterial effect.

Ag nanoparticles, which are preferred in medical applications due to their strong antimicrobial effect, have potential risks on human health such as the release of Ag ions due to their oxidative activities, causing damage to the mitochondrial membrane and causing cytotoxicity through apoptosis, which is called cell death. Youssef et al. The potential cytotoxic effects of the use of Ag nanoparticles in dental treatments in oral applications were investigated; It was concluded that Ag ions induce cytotoxic effect on adult rat pulp tissue. In addition to these, many metal nanomaterials used as antibacterial and antiviral can show genotoxic behavior due to their negative effects on genetic materials, and their usage amounts are important. Although ZnO nanoparticles have effective antimicrobial properties, it is stated in the literature that they tend to form clusters in the apolar polymeric matrix due to their high polarity. In a study conducted by adding ZnO nanoparticles in different weight ratios to the low density polyethylene and ethylene vinyl acetate polymer mixture (LDPE/EVA), it was found that the polymer mixture gained antibacterial properties, but the addition of ZnO nanoparticles to the polymer mixtures formed clusters that prevented mechanical load transfer. It is stated that there is a decrease in the modulus and tensile strength values.

In a different study, the antibacterial behavior of the TPU-based composite formed by adding Ag, TiO₂ and chitosan to the thermoplastic polyurethane was investigated. It was stated that the addition of TiO₂ to the polymer matrix caused a significant decrease in the modulus of elasticity and tensile stress. It was concluded that TiO₂, which exhibits photocatalytic activity, causes deterioration in the crystal structure of the polymer, due to the formation of free radicals (for example, hydroxyl radicals) that can break down the polymer matrix.

### SUMMARY

The invention provides an agent, which provides all kinds of polymer-based appliances, plaques, and transparent or colored retainers used in a dental application with improved mechanical properties and an antibacterial effect. The agent includes at least one of the following materials:
calcium phosphate-based materials, comprising hydroxyapatite (HA), tricalcium phosphate (TCP), biphasic calcium phosphate (BCP), octacalcium phosphate (OCP), brushite, and monetite,
boron based materials, comprising boron (B), boric acid (H₃BO₃), boron oxide (B₂O₃), and boron nitride (BN),
graphene-based materials, comprising graphene, graphene oxide (GO), and reduced graphene oxide (rGO),
magnesium-based materials, comprising magnesium (Mg), magnesium oxide (MgO), magnesium peroxide (MgO₂), and magnesium fluoride (MgF₂),
strontium-based materials, comprising strontium (Sr), strontium oxide (SrO), and strontium hydroxide (Sr(OH)₂),
selenium-based materials, comprising selenium (Se), selenite (SeO₃), selenate (SeO₄), sodium selenite (Na₂SeO₃), and sodium selenate (Na₂SeO₄).

Further, the calcium phosphate-based materials, the boron based materials, the graphene-based materials, the magnesium-based materials, the strontium-based materials, and selenium-based materials each are included as a powder, a suspension, or a solution.

### DETAILED DESCRIPTION OF THE EMBODIMENTS

In dental applications, polymer-based appliances, holders and plaques are used to correct tooth deformities. Tooth deformities occur due to genetic or environmental reasons, and if they are not treated, they cause problems in terms of oral hygiene, oral and jaw health, as well as negatively affect patients psychologically and socially. After orthodontic treatment for the elimination of tooth deformities, appliances, retainers and plaques are used to protect the new position of the corrected teeth and to keep them intact. The treatment, which is generally applied for aesthetic purposes, is now applied due to the necessity of oral and jaw health with the awareness of the patients. In orthodontic treatment, the tendency of the teeth to return to their pre-treatment position is an important problem. Retention is an important stage of orthodontic treatment in which tooth movements are stabilized after active treatment. After the completion of orthodontic treatment, the teeth tend to return to their initial positions due to the tension in the periodontal fibers. Therefore, retention, which is considered the last stage of treatment, is necessary to maintain the desired position of the teeth. The mechanical properties and strength of the appliances, retainers and plates used to prevent the teeth from returning to their former positions until the skeletal growth, gingival and periodontal arrangement is completed. Factors such as temperature, humidity and elastic deformation have a significant effect on the mechanical properties of polymeric materials. Therefore, the mechanical properties of these materials may differ between the intraoral environment and room temperature. However, exposure of polymers to water may cause swelling of the material due to water absorption and may affect the performance of the appliance, holder or plate being used. In addition, depending on the glass transition temperature of the polymer, the material is brittle or ductile at ambient temperature; This affects the mechanical properties.

The invention is used in polymeric dental products, calcium phosphate-based ceramic powders such as hydroxyapatite (HA), tricalcium phosphate (TCP), biphasic calcium phosphate (BCP), octacalcium phosphate (OCP), brushite (bruchite), monetite (monetite) or suspensions containing these powders. or solutions; boron (B) based powders such as boric acid (H₃BO₃), boron oxide (B₂O₃), boron nitride (BN) or suspensions or solutions containing these powders; graphene derivative powders such as graphene, graphene oxide, reduced graphene oxide, or suspensions or solutions containing these powders; Mg-derived powders such as magnesium (Mg), magnesium oxide (MgO), magnesium peroxide (MgO₂), magnesium fluoride (MgF₂) or suspensions or solutions containing these powders; Sr derivative powders such as strontium (Sr), strontium oxide (SrO), strontium hydroxide (Sr(OH)₂) or suspensions or solutions containing these powders; It relates to the material formed by the use of selenium (Se) or Se derivative powders or suspensions or solutions containing these powders, alone or as a mixture.

With the patented material in powder, solution or suspension to be used in the production, coating, surface treatment or content of all kinds of polymer-based appliances, plaques and retainers used in dental applications, mechanical strength will be increased, and polymeric dental products provide improved mechanical properties.

Antibacterial effect will be achieved with the patented coating material in powder, solution or suspension to be used in the production, coating, surface treatment or content of all kinds of polymer-based appliances, plaques and retainers used in dental applications. In addition to mechanical strength, antibacterial effect is also important in polymer-based appliances, holders and plates used in dental applications. The minerals in the tooth enamel are in a natural balance, demineralization and remineralization, and the microbial layer formed on the teeth disrupts the mineral balance in the tooth. The formation of dental caries takes place between the biofilm formed by bacterial colonies and the tooth enamel surface. Bacteria such as S. Mutans on the tooth surface ferment carbohydrates and cause the formation of acid products such as lactic, acetic and propionic. Thus, the oral environment reaches the critical pH value (pH=5.5) for tooth enamel. This situation causes the dissolution of calcium and phosphorus from hydroxyapatite (HA), which is high in tooth enamel, that is, demineralization; With the realization of demineralization on the enamel surface, a color change (opaque white) occurs in the region. These structures are called white spot lesions, which are the first step in the formation of dental caries.

With the use of polymer-based materials in dentistry treatments, more bacterial plaque formation occurs compared to other dental treatments. Covering the tooth and palate structure in the oral environment; As a result of long-term use of a large-sized material, plaque accumulation increases, areas that are difficult to clean are formed and demineralizations occur accordingly. Long-term use of biomaterials can lead to the adhesion and proliferation of bacteria on their surfaces and, in some cases, the formation of dental plaque (biofilm). Color change and odor formation in polymer-based materials due to dental plaque formation are among the most common problems in patient use. Drug treatment or antibiotic-laden biomaterials are used to prevent biofilm formation. However, due to their nature, bacteria can be resistant to antimicrobial drugs as a result of mutation or gene transfer from other organisms.

In order to overcome antibiotic resistance and prevent biofilm formation, the use of antibacterial agents and/or nanoparticles with antibacterial activity is a good alternative to obtain antibacterial biomaterials. Destroying bacteria is related to bacterial variant and antibacterial mechanism of nanoparticles. Bacterial cell wall structure plays an important role against bacterial susceptibility when exposed to nanoparticles. Nanoparticles can adhere to the bacterial wall through electrostatic interaction, disrupt membrane integrity, or cause the formation of “reactive oxygen species” (ROS), which can penetrate cells and cause biomolecules to deteriorate. Nanoparticles are very prone to reactivity and to form ROS due to their large surface area. This physical change, which gives antimicrobial properties, can also lead to high oxidation and DNA destruction in bacteria, depending on the dose increase of the nanoparticle.

In the literature, there are studies on the use of materials such as silver, zinc, hydroxyapatite, boron, magnesium, graphene, strontium, selenium or their derivatives in medical applications (mostly in the coating of metal implants) due to their antibacterial activities. However, studies on obtaining antibacterial activity in dental applications using polymer-based materials such as appliances, plaques and retainers are limited, and most of them contain silver (Ag).

The invention is intended for all kinds of polymer-based appliances, plaques and retainers (transparent or colored) used in dental applications to have antibacterial effect and improved mechanical properties and resistance; It includes the use of at least one of the materials such as hydroxyapatite, boron, graphene, magnesium, strontium, selenium in their production, coating, surface treatments and/or contents.

Hydroxyapatite (HA), which is one of the materials that we will use as an additive material in the production phase of all kinds of polymer-based appliances, plaques and retainers used in dental applications or to form a film layer on their surfaces, is also used in dentistry applications to stop the formation of white spot lesions and to support remineralization. is used. The effect of nano-HA (n-HA) solutions with different concentrations on the initial lesions on enamel was determined; The effect of n-HA as a remineralizing agent was confirmed by providing a caries process similar to the intraoral medium. In a similar study, it was determined that the addition of HA nanoparticles to dental composites increased remineralization on enamel by oscillating in cariogenic environments (pH≤5.5).

Hydroxyapatite (HA), tricalcium phosphate (TCP), biphasic calcium phosphate (BCP), octacalcium phosphate (OCP), brushite, which is one of the material group that we will use to improve the mechanical strength of all kinds of polymer-based appliances, plaques and retainers used in dental applications. Calcium phosphate-based ceramics such as monetite (monetite) are used in medical applications due to their high biocompatibility. These materials can be used without additives within the scope of the patent in order to increase the mechanical strength, or they can also be used with additives with graphene, boron, magnesium, selenium, strontium and their derivatives in order to increase the antibacterial effect.

Boron (B) is an element found in trace amounts in the human body; It is stated that 241 μg B/L in blood and 1130 μg B/L in urine. Although inhalation, dermal and oral toxicity studies indicate that toxicity is observed when the B concentration is high enough; Many in vitro and in vivo studies show that B-containing compounds are not genotoxic. B nanoparticles exhibit an antibacterial effect without the need for any stimulation; They bind to the bacterial cell wall with electrostatic or hydrophobic interactions and disrupt the integrity of the bacteria. There are many studies in the literature about the antibacterial mechanism of boron. In the study examining the antibacterial effect of the mixture of polyhydroxyalkanate (PHA) and hexagonal boron nitride (hBN) with different additive ratios; It is stated that the viability of bacteria decreased significantly due to the increase in the percentage of hBN in the composition. In a different study, it was stated that the antibacterial effect of boron oxide (B₂O₃) addition on the composite structure was caused by the release of B₂O₃ (BO₃)⁻³ ions. After the B ions bind to the hydroxyl group of the lipopolysaccharides in the cell membrane of the bacteria, the viability of the bacteria is terminated, so the composite material can gain antibacterial properties.

Due to their mechanical, electronic, thermal, biological and optical properties, graphene and its derivatives are important for use in medical applications as well as in various applications such as long-lasting batteries, efficient solar cells, circuit boards, display panels. Due to their biocompatibility and rapid functionalization effect, graphene and its derivatives (graphene oxide, reduced graphene oxide; undoped and doped with various elements) are used in the medical sector in the fields of “tissue engineering, molecular drug delivery, cancer therapy, biosensors and bioimaging”, as well as bone repair or organ Studies are carried out for their use in the field of regeneration. In order to increase the antibacterial effect, studies on the use of graphene and its derivatives generally with metal (such as Ag, Cu, Zn), metal oxide (such as TiO₂, ZnO, Fe₃O₄) or metal sulfate are available in the literature. Graphene and its derivatives are included in the invention in order to increase the mechanical strength as well as the antibacterial effect.

Magnesium (Mg) is biodegradable, and the dissolution of Mg and its derivatives in the body results in an alkaline microenvironment that can withstand bacteria effectively. Magnesium oxide nanoparticle (nMgO) is a light metal-based antimicrobial nanoparticle that can be metabolized and fully absorbed in the body. It is reported in the literature that MgO nanoparticles inhibit gram positive, gram negative and endospore forming bacteria. Since nMgO can be efficiently degraded and metabolized in the body (the decomposition products of nMgO and Mg²⁺ and OH⁻ ions can be effectively eliminated from the body as long as kidney function is normal, thus eliminating the concern of excessive metal accumulation in the body), heavy metals such as silver and ZnO It is an attractive alternative to metal-based nanoparticles. Due to the antibacterial effect mentioned in the literature, Mg derivative nano and submicron powders such as magnesium (Mg), magnesium oxide (MgO), magnesium peroxide (MgO₂), magnesium fluoride (MgF₂) or suspensions or solutions containing these powders are included in the invention.

Other materials included in the invention in terms of antibacterial effect are selenium derivatives [selenium (Se), selenite (SeO₃), selenate (SeO₄), sodium selenite (Na₂SeO₃), sodium selenate (Na₂SeO₄)] and strontium derivatives [strontium (Sr), strontium oxide]. (SrO), strontium hydroxide (Sr(OH)₂)]. Selenium (Se) is an important nutrient found in biological tissues, and it is stated in the literature that biomaterials containing Se delay bone tumors and improve the biological activities of tissues. Se is also an antioxidant, protecting the human body against free radicals and carcinogens. Strontium (Sr) is an alkaline earth metal used for the treatment of osteoporosis. Biomaterials containing Sr have been proven to improve bone formation and/or remodeling. It is reported that Sr also accelerates osteogenesis and mineralization. In the literature, it is stated that Se and Sr additives added to hydroxyapatite show excellent antibacterial activity against both gram-negative (escherichia coli) and gram-positive (staphylococcus carnosus) bacterial strains.

Within the scope of the patent; Ca—P, B, graphene, Mg, Se, Sr or their derivatives, nano-or sub-micron-sized powders, suspensions or solutions containing these powders, all kinds of polymer-based appliances, plaques and holders used in dental applications provide antibacterial effect and improve their mechanical properties. is developing.

What is claimed is:

1. An agent, wherein the agent provides all kinds of polymer-based appliances, plaques, and transparent or colored retainers used in a dental application with improved mechanical properties and an antibacterial effect, and the agent comprises at least one of the following materials:
calcium phosphate-based materials, comprising hydroxyapatite (HA), tricalcium phosphate (TCP), biphasic calcium phosphate (BCP), octacalcium phosphate (OCP), brushite, and monetite,
boron based materials, comprising boron (B), boric acid (H₃BO₃), boron oxide (B₂O₃), and boron nitride (BN),
graphene-based materials, comprising graphene, graphene oxide (GO), and reduced graphene oxide (rGO),
magnesium-based materials, comprising magnesium (Mg), magnesium oxide (MgO), magnesium peroxide (MgO₂), and magnesium fluoride (MgF₂),
strontium-based materials, comprising strontium (Sr), strontium oxide (SrO), and strontium hydroxide Sr(OH)₂),
selenium-based materials, comprising selenium (Se), selenite (SeO₃), selenate (SeO₄), sodium selenite (Na₂SeO₃), and sodium selenate (Na₂SeO₄).

2. The agent according to claim 1, comprising the calcium phosphate-based materials as a powder, wherein the calcium phosphate-based materials comprise at least one of the hydroxyapatite (HA), the tricalcium phosphate (TCP), the biphasic calcium phosphate (BCP), the octacalcium phosphate (OCP), the brushite, and the monetite.

3. The agent according to claim 1, comprising the calcium phosphate-based materials as a suspension, wherein the calcium phosphate-based materials comprise at least one of the hydroxyapatite (HA), the tricalcium phosphate (TCP), the biphasic calcium phosphate (BCP), the octacalcium phosphate (OCP), the brushite, and the monetite.

4. The agent according to claim 1, comprising the calcium phosphate-based materials as a solution, wherein the calcium phosphate-based materials comprise at least one of the hydroxyapatite (HA), the tricalcium phosphate (TCP), the biphasic calcium phosphate (BCP), the octacalcium phosphate (OCP), the brushite, and the monetite.

5. The agent according to claim 1, comprising the boron based materials as a powder, wherein the boron based materials comprise at least one of the boron (B), the boric acid (H₃BO₃), the boron oxide (B₂O₃), and the boron nitride (BN).

6. The agent according to claim 1, comprising the boron based materials as a suspension, wherein the boron based materials comprise at least one of the boron (B), the boric acid (H₃BO₃), the boron oxide (B₂O₃), and the boron nitride (BN).

7. The agent according to claim 1, comprising the boron based materials as a solution, wherein the boron based materials comprise at least one of the boron (B), the boric acid (H₃BO₃), the boron oxide (B₂O₃), and the boron nitride (BN).

8. The agent according to claim 1, comprising the graphene-based materials as a powder, wherein the graphene-based materials comprise at least one of the graphene, the graphene oxide (GO), and the reduced graphene oxide (rGO).

9. The agent according to claim 1, comprising the graphene-based materials as a suspension, wherein the graphene-based materials comprise at least one of the graphene, the graphene oxide (GO), and the reduced graphene oxide (rGO).

10. The agent according to claim 1, comprising the graphene-based materials as a solution, wherein the graphene-based materials comprise at least one of the graphene, the graphene oxide (GO), and the reduced graphene oxide (rGO).

11. The agent according to claim 1, comprising the magnesium-based materials as a powder, wherein the magnesium-based materials comprise at least one of the magnesium (Mg), the magnesium oxide (MgO), the magnesium peroxide (MgO₂), and the magnesium fluoride (MgF₂).

12. The agent according to claim 1, comprising the magnesium-based materials as a suspension, wherein the magnesium-based materials comprise at least one of the magnesium (Mg), the magnesium oxide (MgO), the magnesium peroxide (MgO₂), and the magnesium fluoride (MgF₂).

13. The agent according to claim 1, comprising the magnesium-based materials as a solution, wherein the magnesium-based materials comprise at least one of the magnesium (Mg), the magnesium oxide (MgO), the magnesium peroxide (MgO₂), and the magnesium fluoride (MgF₂).

14. The agent according to claim 1, comprising the strontium-based materials as a powder, wherein the strontium-based materials comprise at least one of the strontium (Sr), the strontium oxide (SrO), and the strontium hydroxide (Sr(OH)₂).

15. The agent according to claim 1, comprising the strontium-based materials as a suspension, wherein the strontium-based materials comprise at least one of the strontium (Sr), the strontium oxide (SrO), and the strontium hydroxide (Sr(OH)₂).

16. The agent according to claim 1, comprising the strontium-based materials as a solution, wherein the strontium-based materials comprise at least one of the strontium (Sr), the strontium oxide (SrO), and the strontium hydroxide (Sr(OH)₂).

17. The agent according to claim 1, comprising the selenium-based materials as a powder, wherein the selenium-based materials comprise at least one of the selenium (Se), the selenite (SeO₃), the selenate (SeO₄), the sodium selenite (Na₂SeO₃), and the sodium selenate (Na₂SeO₄).

18. The agent according to claim 1, comprising the selenium-based materials as a suspension, wherein the selenium-based materials comprise at least one of the selenium (Se), the selenite (SeO₃), the selenate (SeO₄), the sodium selenite (Na₂SeO₃), and the sodium selenate (Na₂SeO₄).

19. The agent according to claim 1, comprising the selenium-based materials as a solution, wherein the selenium-based materials comprise at least one of the selenium (Se), the selenite (SeO₃), the selenate (SeO₄), the sodium selenite (Na₂SeO₃), and the sodium selenate (Na₂SeO₄).
